(19) United States
(12) Patent Application Publication
(10) Pub. No.: US 2025/0171857 A1
(43) Pub. Date: May 29, 2025

(54) BIOMARKERS FOR DIAGNOSING OR PREDICTING PROGNOSIS OF NON-INVASIVE FOLLICULAR THYROID NEOPLASM WITH PAPILLARY-LIKE NUCLEAR FEATURES AND METHOD FOR TREATMENT OF THYROID NODULE

(71) Applicant: THE CATHOLIC UNIVERSITY OF KOREA INDUSTRY-ACADEMIC COOPERATION FOUNDATION, Seoul (KR)

(72) Inventors: Chan Kwon JUNG, Seoul (KR); Seon Young KIM, Daejeon (KR); Jong-Lyul PARK, Daejeon (KR); So-Yeon LEE, Daejeon (KR)

(73) Assignee: THE CATHOLIC UNIVERSITY OF KOREA INDUSTRY-ACADEMIC COOPERATION FOUNDATION, Seoul (KR)

(21) Appl. No.: 18/958,993

(22) Filed: Nov. 25, 2024

(30) Foreign Application Priority Data
Nov. 24, 2023 (KR) 10-2023-0165859

(51) Int. Cl. C12Q 1/6886; C12Q 1/6851

(52) U.S. Cl. CPC C12Q 1/6886; C12Q 1/6851; C12Q 2600/158; C12Q 2600/166

(57) ABSTRACT

The present disclosure relates to a novel thyroid nodule diagnostic biomarker and use thereof, and more specifically to an mRNA biomarker or a combination of mRNA biomarkers that are capable of diagnosing non-invasive follicular thyroid neoplasm with papillary-like nuclear features (NIFTP) among thyroid nodules with high accuracy and sensitivity compared to benign and malignant tumors. In the present disclosure, it was confirmed that the expression levels of OCLN, ZNF423, LYG1 and AQP5 are associated with the onset or aggravation of non-invasive follicular thyroid neoplasm with papillary-like nuclear features (NIFTP). Accordingly, the present disclosure provides OCLN, ZNF423, LYG1 and AQP5 genes as diagnostic biomarkers for non-invasive follicular thyroid neoplasm with papillary-like nuclear features (NIFTP).

### CROSS-REFERENCE TO RELATED APPLICATION(S)

This application claims priority to and the benefit of Korean Patent Application No. 10-2023-0165859, filed on Nov. 24, 2023, the disclosure of which is incorporated herein by reference in its entirety.

### TECHNICAL FIELD

The present disclosure has confirmed that the onset of non-invasive follicular thyroid neoplasm with papillary-like nuclear features (NIFTP) is related to the expression levels of OCLN, ZNF423, LYG1 and AQP5 genes, and provides OCLN, ZNF423, LYG1 and AQP5 genes as biomarkers for diagnosing NIFTP. By using the biomarker composition, kit and method of the present disclosure, it is possible to easily determine whether NIFTP has occurred, predict a prognosis thereof or provide a method for treatment.

### BACKGROUND

Follicular cells, which make up the majority of normal thyroid tissue, form follicular structures (also called ‘follicles’). Tumors that originate from these follicular cells and form follicular structures include follicular adenoma (FA), non-invasive follicular thyroid neoplasm with papillary-like nuclear features (NIFTP), follicular thyroid carcinoma (FTC) and infiltrative follicular variant of papillary thyroid carcinoma (PTC). Non-invasive follicular thyroid neoplasm with papillary-like nuclear features (NIFTP) and infiltrative follicular variant of PTC show the characteristic nuclear shape of papillary carcinoma, unlike follicular adenoma and follicular carcinoma. Follicular neoplasm is used as an umbrella term for follicular adenoma, NIFTP, FTC, and follicular variant of PTC.

PTC has about 10 variants or subtypes, and the most common subtype is follicular variant PTC (FVPTC), which accounts for about 10 to 20% of all PTCs. FVPTC can be further classified into encapsulated FVPTC (EFVPTC) and infiltrative FVPTC. Encapsulated follicular variant of PTC (EFVPTC) can also be further classified into invasive (invasive EFVPTC) and non-invasive (non-invasive EFVPTC) based on the presence or absence of invasion of the tumor capsule and blood vessels. In cases with a capsule, it often shows the findings of a follicular tumor with an isoechoic, homogeneous tumor and a hollow surrounding the tumor. Pathologically, FVPTC can be defined as a tumor that has the nuclear shape of classic PTC but has a follicular structure, that is, a cytopathological structure similar to a follicular tumor. Infiltrative FVPTC shows the same prognosis as classic PTC. However, it is well known that non-invasive EFVPTC shows an extremely good prognosis, similar to a benign tumor.

Professor Yuri Nikiforov's team at the University of Pittsburgh in the United States reported that there was no recurrence or death during the follow-up period (10-26 years, median 13 years) after surgery among 109 patients with non-invasive EFVPTC, and renamed non-invasive EFVPTC to NIFTP and suggested that it should no longer be classified as cancer, and further presented new pathological diagnostic guidelines. Starting with the revised World Health Organization (WHO) Endocrine Tumors Classification Book in 2017, NIFTP began to be classified as a borderline thyroid tumor (low-risk neoplasm) rather than a malignant tumor.

With current technology, NIFTP can only be diagnosed by a detailed examination of the entire tumor through postoperative pathology examination, sometimes accompanied by immunohistochemical or molecular pathology examination. In order to diagnose, surgical tumor removal is a prerequisite. In other words, in order to reduce unnecessary surgery, NIFTP should be diagnosed through fine needle aspiration cytology or core needle biopsy of thyroid nodules performed before surgery, but such a method is currently unknown. In most studies, nodules ultimately diagnosed as NIFTP were diagnosed as the Bethesda category III, IV or V through preoperative cytology or biopsy, and 18% to 27% were diagnosed as suspicious for PTC.

Therefore, there is a need to develop a biomarker that can reduce the overdiagnosis of thyroid cancer and differentiate NIFTP downgraded from malignant to borderline tumor for more detailed diagnosis, thereby preventing unnecessary treatment and alleviating patients' concerns about cancer.

### SUMMARY OF THE INVENTION

The present disclosure has been devised to solve the above problems and meet the above needs, and an object of the present disclosure is to provide biomarkers for confirming changes in the gene expression level of cells in nodules in patients with thyroid nodules and determining non-invasive follicular thyroid neoplasm with papillary-like nuclear features (NIFTP) among thyroid tumors.

Additionally, the present disclosure provides a biomarker composition that can diagnose the occurrence of non-invasive follicular thyroid neoplasm with papillary-like nuclear features (NIFTP) and predict a prognosis thereof.

In addition, the present disclosure provides a kit that can diagnose non-invasive follicular thyroid neoplasm with papillary-like nuclear features (NIFTP).

In order to solve the above-described problems, the present disclosure provides a biomarker composition for diagnosing or predicting a prognosis of non-invasive follicular thyroid neoplasm with papillary-like nuclear features (NIFTP), including OCLN, ZNF423, LYG1, AQP5 or a combination thereof.

If the expression of the OCLN or ZNF423 gene increases (upregulation) or the expression of the LYG1 or AQP5 gene decreases (downregulation) in a sample isolated from a subject, it may be predicted or diagnosed that non-invasive follicular thyroid neoplasm with papillary-like nuclear features (NIFTP) has developed.

In addition, the present disclosure provides a composition for diagnosing or predicting a prognosis of non-invasive follicular thyroid neoplasm with papillary-like nuclear features (NIFTP), including an agent for measuring an expression level of OCLN, ZNF423, LYG1 or AQP5 gene.

Additionally, the composition for diagnosing or predicting a prognosis of non-invasive follicular thyroid neoplasm with papillary-like nuclear features (NIFTP), the agent for measuring the expression of the gene may include a peptide, antibody or primer that specifically binds to the gene.

In addition, the present disclosure provides a kit for diagnosing or predicting a prognosis of non-invasive follicular thyroid neoplasm with papillary-like nuclear features (NIFTP), including the composition for diagnosing or predicting a prognosis of non-invasive follicular thyroid neoplasm with papillary-like nuclear features (NIFTP).

In another embodiment of the present disclosure, provided is a method for diagnosing or predicting a prognosis of non-invasive follicular thyroid neoplasm with papillary-like nuclear features (NIFTP), the method including: a) measuring an expression level of OCLN, ZNF423, LYG1 or AQP5 gene in a sample isolated from a subject; b) comparing the expression level of the OCLN, ZNF423, LYG1 or AQP5 gene with an expression level of a normal control sample; and c) determining that non-invasive follicular thyroid neoplasm with papillary-like nuclear features (NIFTP) has developed if the expression level of the OCLN, ZNF423, LYG1 or AQP5 gene measured in the sample isolated from the subject is different from the expression level of the normal control sample.

Additionally, the expression level of the OCLN, ZNF423, LYG1 or AQP5 gene may be measured by at least any one method selected from the group consisting of Western blot, enzyme-linked immunosorbent assay (ELISA), immunohistochemical staining, immunoprecipitation, immunofluorescence, transcriptome, epigenome and quantitative real-time PCR techniques.

Additionally, the sample isolated in step a) may include saliva, urine, tissue, whole blood, serum or plasma, but is not limited thereto.

In another embodiment of the present disclosure, provided is a method for determining a treatment option for a patient with thyroid tumor, the method including: a) measuring an expression level of OCLN, ZNF423, LYG1 or AQP5 gene from a sample isolated from a patient with thyroid tumor; b) comparing the expression level of the gene with an expression level of a normal control sample; and c) determining that a thyroid nodule is non-invasive follicular thyroid neoplasm with papillary-like nuclear features (NIFTP), if the expression level of the OCLN or ZNF423 gene measured in the sample isolated from a thyroid nodule patient increases or the expression level of the LYG1 or AQP5 gene decreases.

And Also, provide a treatment method for a patient with thyroid tumor, the method comprising: a) measuring an expression level of OCLN, ZNF423, LYG1 or AQP5 gene from a sample isolated from a patient with thyroid tumor; b) comparing the expression level of the gene with an expression level of a normal control sample; c) determining that a thyroid nodule is non-invasive follicular thyroid neoplasm with papillary-like nuclear features (NIFTP), if the expression level of the OCLN or ZNF423 gene measured in the sample isolated from a thyroid nodule patient increases or the expression level of the LYG1 or AQP5 gene decreases; and d) Administering non-invasive follicular thyroid neoplasm with papillary-like nuclear features (NIFTP) treatment drugs to patients diagnosed with non-invasive follicular thyroid neoplasm with papillary-like nuclear features (NIFTP).

The present disclosure analyzed the genes of patients with non-invasive follicular thyroid neoplasm with papillary-like nuclear features (NIFTP) and selected genes that show a specific expression pattern of non-invasive follicular thyroid neoplasm with papillary-like nuclear features (NIFTP) among thyroid tumors. Accordingly, in the present disclosure, non-invasive follicular thyroid neoplasm with papillary-like nuclear features (NIFTP) can be diagnosed with high specificity by using the above genes as biomarkers.

In addition, the present disclosure provides a combination of biomarkers that are capable of diagnosing the occurrence of non-invasive follicular thyroid neoplasm with papillary-like nuclear features (NIFTP) and calculation formulas for diagnosing the occurrence of NIFTP using the same (see Formula 1 and Formula 2 below).

When the biomarker composition of the present disclosure is used, it is possible to non-invasively and quickly diagnose the occurrence of NIFTP.

### BRIEF DESCRIPTION OF THE DRAWINGS

FIG. 1A and FIG. 1B are mRNA expression profiling images of fresh frozen thyroid tumor tissues (n=74). FIG. 1A is an unsupervised hierarchical clustering analysis to investigate the expression levels of 255 genes downregulated and 255 genes upregulated in NIFTP and malignant tumors, which were confirmed identically using edgeR software. In FIG. 1A, RNA expression levels were normalized to Z-scores.

FIG. 1B is the result of KEGG pathway enrichment analysis for differentially expressed genes (DEGs) between benign thyroid tumors and NIFTP/malignant thyroid tumors. Malignant thyroid tumors included papillary carcinoma and follicular carcinoma.

FIG. 2A and FIG. 2B are the results of identifying NIFTP-specific mRNA markers in the discovery dataset of the present disclosure. FIG. 2A shows the results of comparative analysis of differentially expressed mRNAs between NIFTP and other subtypes [follicular nodular disease (FND), follicular adenoma (FA), follicular thyroid carcinoma (FTC) and papillary thyroid carcinoma (PTC)], and it was found that there were 19 upregulated mRNAs and 7 downregulated mRNAs in NIFTP. FIG. 2B is a heat map of differentially expressed mRNAs for thyroid tumor subgroups (in FIG. 2B, RNA expression levels were not Z-score normalized).

FIG. 3A and FIG. 3B show the results of multiple mRNA marker selection for the identification of NIFTP in the discovery dataset of the present disclosure. FIG. 3A shows the results of AIC (Akaike information criterion) analysis using 12 candidate mRNA markers to distinguish NIFTP with the Blorr R software package. The genes selected by AIC included OCLN, ZNF423, LYG1 and AQP5. FIG. 3B shows the results of analyzing the expression patterns of 12 mRNA markers selected from each thyroid tumor using RNA expression levels normalized to log 2 CPM. ANOVA analysis was used to estimate statistical significance.

FIGS. 4A and 4B show the results of receiver operating characteristic (ROC) analysis of a multiple logistic regression model for distinguishing NIFTP from other types of thyroid tumors in the discovery dataset and validation dataset.

FIG. 5 shows the results of analyzing the expression levels of 4 mRNA markers selected from the validation dataset using qRT-PCR according to one embodiment of the present disclosure. In FIG. 5, the expression levels of OCLN (A), ZNF423 (B), LYG1 (C) and AQP5 (D) are shown in relation to normal thyroid tissue and various tumor types. In FIG. 5, FND stands for thyroid follicular nodular disease, FA stands for follicular adenoma, NIFTP stands for non-invasive follicular thyroid neoplasm with papillary-like nuclear features, IEFVPTC stands for invasive encapsulated follicular variant of papillary thyroid carcinoma, FTC stands for follicular thyroid carcinoma, and PTC stands for papillary thyroid carcinoma.

FIG. 6 shows the results of comparing NIFTP vs PTC, NIFTP vs FTC, NIFTP vs FA and NIFTP vs FND of the top 12 candidate mRNA markers for distinguishing NIFTP from other thyroid tumors in the discovery dataset of the present disclosure.

### DETAILED DESCRIPTION OF THE EMBODIMENTS

Hereinafter, the present disclosure will be described in detail. The advantages and features of the present disclosure and the embodiments that achieve the same will be apparent with reference to the embodiments described below. However, the present disclosure is not limited to the embodiments disclosed below, but can be implemented in various different forms, and these embodiments are provided only to make the disclosure of the present disclosure complete and to fully inform those skilled in the art to which the present disclosure pertains of the scope of the invention, and the present disclosure is defined only by the scope of the claims. Like reference numerals throughout the specification refer to like elements.

Unless otherwise defined, all terms (including technical and scientific terms) used in the present specification may be used in meanings that can be commonly understood by those skilled in the art to which the present disclosure pertains. In addition, terms defined in commonly used dictionaries shall not be ideally or excessively interpreted unless explicitly specifically defined. The terms used in the present specification are for the purpose of describing the embodiments and are not intended to limit the present disclosure. In the present specification, singular forms also include plural forms unless specifically stated in the phrase.

The present disclosure has confirmed that the expressions of KCNC1, PDE1B, NXPH4, ZNF423, MAPT, SRMS, OCLN, IGF2BP3, AQP5, LYG1, IDO1 and PHYHD1 mRNA are related to non-invasive follicular thyroid neoplasm with papillary-like nuclear features (NIFTP), thereby completing the present disclosure.

According to the present disclosure, when 8 (KCNC1, PDE1B, NXPH4, ZNF423, MAPT, SRMS, OCLN and IGF2BP3) of the above 12 mRNAs are upregulated or 4 (AQP5, LYG1, IDO1 and PHYHD1) are downregulated, NIFTP may be diagnosed as having developed.

The present disclosure provides a biomarker composition for diagnosing or predicting a prognosis of non-invasive follicular thyroid neoplasm with papillary-like nuclear features (NIFTP), including, particularly among the above 12 mRNA markers, occludin (OCLN), zinc Finger Protein 423 (ZNF423), lysozyme g1 (LYG1), aquaporin 5 (AQP5) or a combination thereof.

In the case of the present disclosure, based on the expression levels of occludin (OCLN), zinc Finger Protein 423 (ZNF423), lysozyme g1 (LYG1) and aquaporin 5 (AQP5) genes measured in a total RNA sample isolated from a subject, a prediction score is obtained through Formula 1 or Formula 2 below, and if the score is 23.872 or 1.374 or higher, respectively, NIFTP may be diagnosed as having developed.

### <Formula 1>

When analyzing the gene expression level using next-generation sequencing (mRNA-seg) targeting nucleic acids extracted from fresh tissue samples

$$Prediction score = - 25.12 + ( 3.797 \times expression level of OCLN ) + ( 3.69 \times expression level of ZNF 423 ) + ( - 5.229 \times expression level of LYG 1 ) + ( - 0.756 \times expression level of AQP 5 ) .$$

### <Formula 2>

When qRT-PCR analysis is performed on nucleic acids extracted from formalin-fixed paraffin-embedded tissue blocks,

$$Prediction score = - 3.722 + ( 9.95 \times expression level of OCLN ) + ( 70.667 \times expression level of ZNF 423 ) - ( 273.325 \times expression level of LYG 1 ) + ( 2.752 \times expression level of AQP 5 ) .$$

The biological sample may include whole blood, plasma, serum, red blood cells, white blood cells (e.g., peripheral blood monocytes), ductal fluid, ascites, pleural efflux, nipple aspirate, lymph (e.g., disseminated tumor cells in a lymph node), bone marrow aspirate, saliva, urine, feces (i.e., excretions), sputum, bronchial washings, tears, fine needle aspirates, any other bodily fluid, tissue samples (e.g., tumor tissue), tumor biopsies (e.g., puncture biopsies), lymph nodes (e.g., sentinel lymph node biopsies), surgical resections of tumors and cellular extracts thereof, and preferably, it may be plasma.

The biomarkers of the present disclosure may be detected by a known method for measuring the expression level and activity of glutathione expression, such as Western blot, enzyme-linked immunosorbent assay (ELISA), immunohistochemical staining, immunoprecipitation or immunofluorescence, and the expression level may be analyzed by analyzing the same using a machine learning technique.

In the present disclosure, the biological sample may be preprocessed to detect the biomarkers. For example, it may include the filtration, distillation, extraction, separation, concentration, inactivation of interfering components, addition of reagents and the like.

As used herein, the term “quantitative device” means a device that provides quantitative numerical information on the presence or absence of a specific biomarker in a biological sample as well as the relative or absolute amount thereof. Specifically, the quantitative device is chromatography, mass spectroscopy (MS) or nuclear magnetic resonance (NMR).

As used herein, the term “chromatography” includes high performance liquid chromatography (HPLC), liquid-solid chromatography (LSC), paper chromatography (PC), thin-layer chromatography (TLC), gas-solid chromatography (GSC), liquid-liquid chromatography (LLC), foam chromatography (FC), emulsion chromatography (EC), gas-liquid chromatography (GLC), ion chromatography (IC), gel filtration chromatography (GFC) or gel permeation chromatography (GPC), but is not limited thereto, and any quantitative chromatography commonly used in the art may be used.

As used herein, the term “mass spectrometry (MS)” refers to a process of measuring the mass of a target substance to analyze the chemical composition of a sample. Mass spectrometry generates charged molecules or molecular fragments through ionization of the target substance present in the sample, and provides information about the mass by measuring the mass-to-charge ratio (m/z) and the abundance ratio of gaseous ions. Such mass spectrometry devices include, but are not limited to, for example, Matrix-Assisted Laser Desorption/Ionization Time of Flight (MALDI-TOF), Surface Enhanced Laser Desorption/Ionization Time of Flight (SELDI-TOF), Electrospray ionization Time of Flight (ESI-TOF), liquid chromatography-mass spectrometry (LC-MS) or liquid chromatography-Mass Spectrometry/Mass Spectrometry (LC-MS/MS).

As used herein, the term “diagnosis” includes determining the susceptibility of a subject to a particular disease or condition, determining whether a subject currently has a particular disease or condition, determining the prognosis of a subject having a particular disease or condition, or therametrics, for example, monitoring the condition of a subject to provide information about the efficacy of a treatment.

As used herein, the term “prognosis” refers to the course of a disease such as a thyroid tumor (e.g., the possibility of thyroid tumor-induced death or progression (defined as TNM stage progression after disease recurrence) including recurrence, metastatic spread and drug resistance) and whether it is cured. For the purpose of the present disclosure, prognosis refers to predicting the possibility of recurrence or further progression after treatment of a thyroid tumor.

As used herein, the term “prognosis prediction marker”, “marker for predicting a prognosis” or “prognosis marker” refers to a substance that can predict a prognosis, including whether to progress or relapse after the treatment of non-invasive follicular thyroid neoplasm with papillary-like nuclear features (NIFTP) by distinguishing NIFTP cells from normal cells.

As used herein, the term “patient” generally includes a human, but may also include other animals, such as other primates, rodents, dogs, cats, horses, sheep, pigs and the like. The ‘patient’ of the present disclosure includes subjects other than humans that are diagnosed with or suspected of having non-invasive follicular thyroid neoplasm with papillary-like nuclear features (NIFTP).

Hereinafter, in order to help understanding of the present disclosure, examples will be described in detail.

### Example 1

### Materials and Experimental Methods

### 1-1. Experimental Subjects

The content of this example was approved by the Catholic University of Korea Seoul St. Mary's Hospital Clinical Trial Review Board (KC20TISI0766). Thyroid tumor tissue and non-tumor tissue samples used in the present disclosure were collected from the Seoul St. Mary's Hospital Biobank. The demographics and basic characteristics of the patients used in the present disclosure are summarized in Table 1 below.

The inventors of the present disclosure collected 74 fresh frozen tissue samples to profile mRNA expression levels. The samples included 10 cases of thyroid follicular nodular disease (FND), 24 cases of follicular adenoma (FA), 14 cases of non-invasive follicular thyroid neoplasm with papillary-like nuclear features (NIFTP), 6 cases of classic papillary thyroid carcinoma (PTC), 6 cases of other PTC subtypes (3 cases of encapsulated classic PTC with predominant follicular growth, 2 cases of invasive encapsulated solid/trabecular PTC, and 1 case of diffuse sclerosing PTC), and 14 cases of follicular thyroid carcinoma (FTC). All tumor samples used in the experiments of the discovery dataset were deliberately selected to be negative for BRAF V600E mutations to facilitate the discovery of NIFTP-specific genes.

TABLE 1

| Feature | Discovery Dataset | | Validation Dataset | |
|---|---|---|---|---|
| Number of samples | 74 | | 90 | |
| Age at diagnosis, mean (range) | 48 | (19-78) | 50 | (19-80) |

Gender

| Female | 50 | | 73 | |
|---|---|---|---|---|
| Male | 24 | | 17 | |
| Tumor size (cm), mean (range) | 3.0 | (1.4-6.0) | 3.2 | (1.2-12.0) |

Diagnosis

| | | |
|---|---|---|
| Normal thyroid tissue | 0 | 12 |
| Follicular nodular disease | 10 | 13 |
| Follicular adenoma | 24 | 18 |
| NIFTP | 14 | 10 |
| IEFVPTC, minimally invasive | 0 | 14 |
| PTC, classic | 6 | 6 |
| PTC, encapsulated classic | 4 | 3 |
| PTC, tall cell | 0 | 3 |
| PTC, diffuse sclerosing | 1 | 1 |
| PTC, encapsulated solid/ | 1 | 0 |
| trabecular | | |
| PTC with BRAF V600E | 0 | 11 |
| PTC with BRAF wild-type | 12 | 2 |
| FTC, minimally invasive | 11 | 7 |
| FTC, encapsulated angioinvasive | 3 | 2 |
| FTC, widely invasive | 0 | 1 |

1-2. Total RNA Preparation and mRNA Sequencing

Total RNA was isolated from fresh-frozen tissues using the RNeasy Kit (Qiagen, Carlsbad, CA, USA) according to the manufacturer's instructions. The quantity and quality of the obtained total RNA were assessed using an ND-1000 spectrophotometer (Thermo Fisher Scientific, Waltham, MA, USA). RNA integrity number (RIN) was estimated using a 2100 Agilent Bioanalyzer (Agilent Technologies, Waldbronn, Germany). Approximately 1 μg of total RNA was utilized for library preparation using the Illumina TruSeq Stranded Total RNA Library Prep Kit (San Diego, CA, USA). 101-bp paired-end sequencing was performed using an Illumina NovaSeq 6000 System sequencing instrument and generated according to the manufacturer's instructions.

1-3. mRNA Sequencing Data Analysis

The inventors of the present disclosure used Trimmomatic software (v. 0.38) to remove TruSeq small RNA adapters from the sequenced reads. The remaining sequence data were mapped to the human genome (GRCh38) for quantification using STAR software (v. 2.7.a). EdgeR software was used to identify differentially expressed mRNAs. The inventors of the present disclosure utilized default parameter configurations for all programs. The mRNA-seq data sets are available from the Korean Nucleotide Archive (KoNA, https://kobic.re.kr/kona) and Sequence Read Archive (SRA, https://www.ncbi.nlm.nih.gov/sra) public databases (Accession Numbers: PRJKA220514 and PRJNA918826).

Examples of genes used in the present disclosure are shown in Table 2 below.

TABLE 2

| Name | Explanation |
|---|---|
| KCNC1 | potassium voltage-gated channel subfamily C member 1 |
| | [Homo sapiens (human)], Gene ID: 3746 |
| PDE1B | phosphodiesterase 1B [Homo sapiens (human)], |
| | Gene ID: 5153 |
| NXPH4 | neurexophilin 4 [Homo sapiens (human)], |
| | Gene ID: 11247 |
| ZNF423 | zinc finger protein 423 [Homo sapiens (human)], |
| | Gene ID: 23090 |
| MAPT | microtubule associated protein tau [Homo sapiens |
| | (human)], Gene ID: 4137 |
| SRMS | src-related kinase lacking C-terminal regulatory tyrosine and |
| | N-terminal myristylation sites [Homo sapiens |
| | (human)], Gene ID: 6725 |
| OCLN | occludin [Homo sapiens (human)], Gene ID: 100506658 |
| IGF2BP3 | insulin like growth factor 2 mRNA binding protein 3 |
| | [Homo sapiens (human)], Gene ID: 10643 |
| AQP5 | aquaporin 5 [Homo sapiens (human)], Gene ID: 362 |
| LYG1 | lysozyme g1 [Homo sapiens (human)], Gene ID: 129530 |
| IDO1 | indoleamine 2,3-dioxygenase 1 [Homo sapiens |
| | (human)], Gene ID: 3620 |
| PHYHD1 | phytanoyl-CoA dioxygenase domain containing 1 |
| | [Homo sapiens (human)], Gene ID: 254295 |

1-4. Analysis of Public mRNA Sequencing Data

In order to determine the mRNA expression patterns of candidate mRNA markers, the inventors of the present disclosure obtained public mRNA sequencing data of thyroid samples from The Cancer Genome Atlas (TCGA) data set (https://portal.gdc.cancer.gov/).

### 1-5. Statistical Analysis

Student's t-test or analysis of variance (ANOVA) was used to evaluate the significance of gene expression differences between benign tumor tissues and NIFTP/malignant tumor tissues. Hierarchical clustering was performed using Multiple Experiment Viewer (MEV) software (version 4.8.1) and the Pearson correlation method. ShinyGO tool was used for Kyoto Encyclopedia of Genes and Genomes (KEGG) Gene Ontology (GO) enrichment analysis. The receiver operating characteristic (ROC) and the area under the ROC curve (AUC) for each mRNA marker were calculated using the ROCR package of R software (version 4.2.2). In addition, the inventors of the present disclosure performed a logistic regression analysis on the mRNA marker combination to identify NIFTP in other thyroid tumors. The prediction score was calculated by multiplying the expression level of each mRNA by its corresponding regression coefficient and then summing the same as a linear combination. The inventors of the present disclosure estimated the optimal cutoff value that maximized the sensitivity and specificity between low and high levels of mRNA expression using ROC curve analysis, and the results with a p-value less than 0.05 were considered as significant.

### Example 2

### Experimental Results

### 2-1. Overall Differential Gene Expression Between Benign and NIFTP/Malignant Thyroid Tumors

In the above experiment, the inventors of the present disclosure performed mRNA expression profiling on 74 fresh-frozen thyroid tissues, including benign thyroid tumors (FND and FA), NIFTP and malignant tumors (PTC and FTC). In the excavation dataset experiment, only PTCs without BRAF V600E mutation were included. In order to identify differentially expressed genes (DEGs), the inventors of the present disclosure applied two criteria: (1) p-value <0.05; and (2) log 2 fold change between benign thyroid tumors (FA and FND) and NIFTP/malignant tumors (FTC and PTC)>0.5.

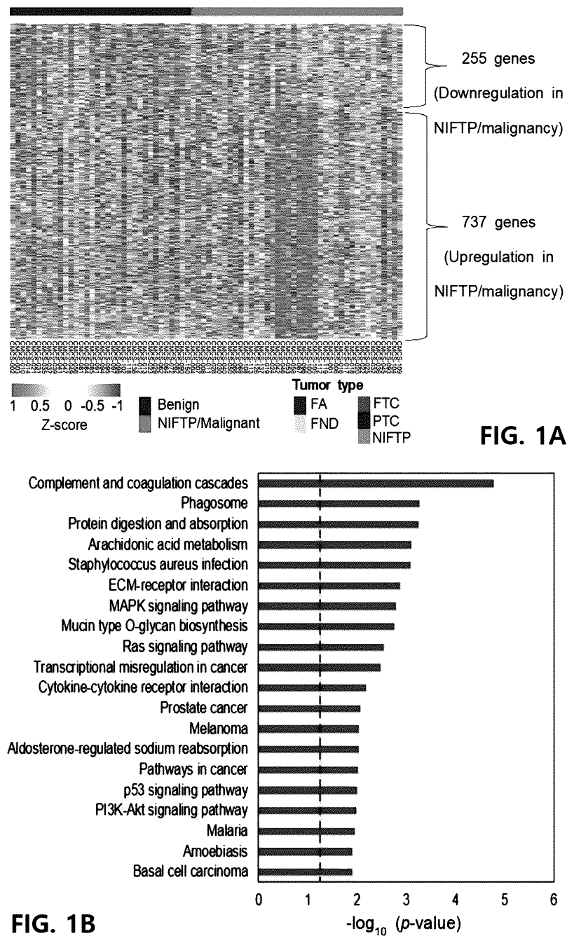

Based on these criteria, the present disclosure identified 255 genes that were downregulated and 737 genes that were upregulated in NIFTP/malignant tumors compared to benign tumors. FIG. 1A shows the results of unsupervised hierarchical clustering using these DEG candidates.

Next, the inventors of the present disclosure performed KEGG pathway enrichment analysis using DEGs between benign tumors and NIFTP/malignant tumors, and discovered several tumor-related pathways, including ECM receptor interaction, MAPK signaling pathway, Ras signaling pathway, cancer pathway and p53 signaling pathway (FIG. 1B). These results demonstrate that the RNA-seq platform can successfully identify DEGs between benign tumors and NIFTP/malignant thyroid tumors.

2-2. Identification of Differentially Expressed mRNAs in NIFTP

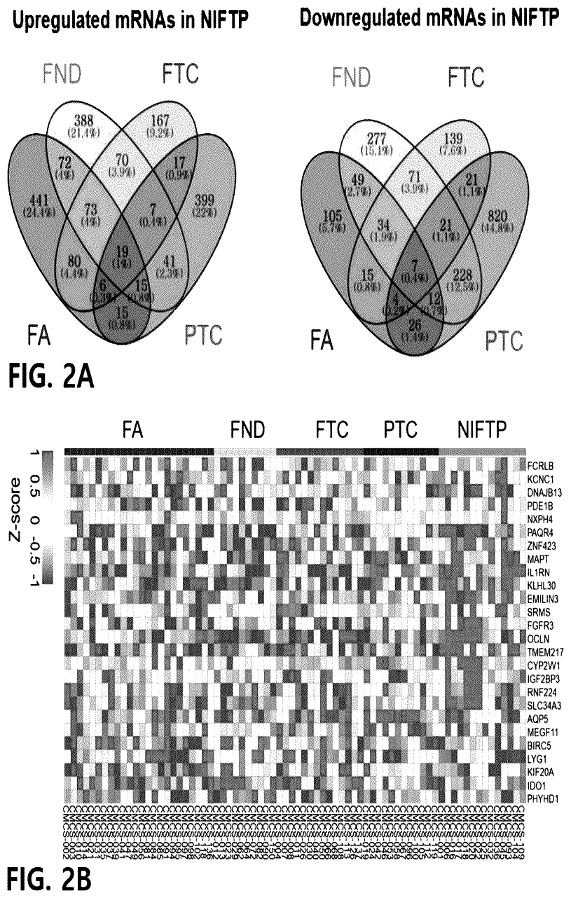

The inventors of the present disclosure also analyzed differentially expressed mRNAs between various tumor subgroups (FND vs NIFTP, FA vs NIFTP, FTC vs NIFTP, PTC vs NIFTP) to identify NIFTP-specific mRNA markers. By using Venn diagrams, the inventors of the present disclosure identified 19 significantly upregulated and 7 significantly downregulated mRNAs in NIFTP (FIG. 2A). Unsupervised hierarchical clustering of candidate mRNA markers was performed, and the results are shown in FIG. 2B.

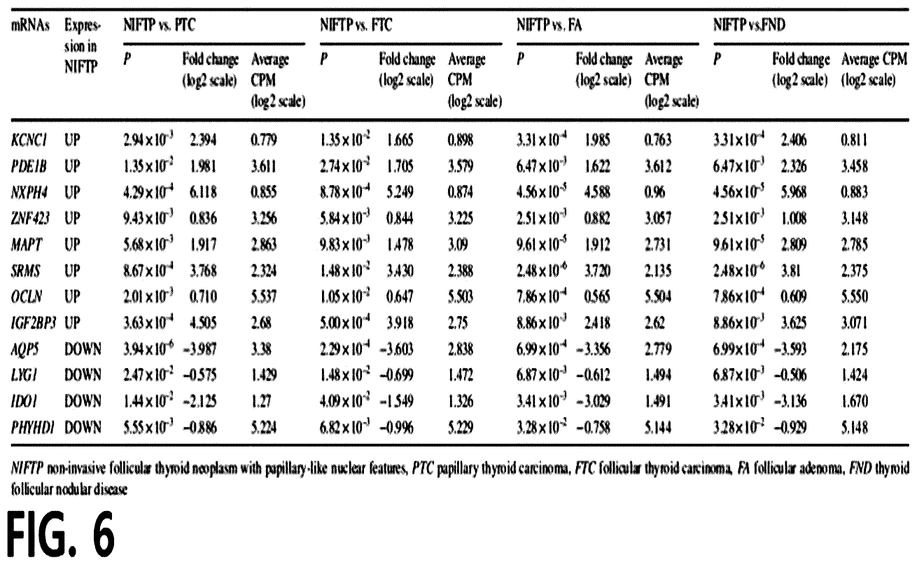

Afterwards, the inventors of the present disclosure analyzed the TCGA data set to further refine the list of NIFTP-specific candidate mRNA biomarkers. The TCGA project did not register NIFTP cases because tumors were not yet recognized as a separate entity until the completion of the TCGA project in 2016. In the present disclosure, the inventors of the present disclosure reviewed the whole slide images available in the TCGA dataset and selected 26 cases that could potentially be classified as NIFTP. Cases with BRAF V600E-like molecular alterations were excluded. Although some of the identified cases may not meet the diagnostic criteria for NIFTP, they still played an important role in validating the results. Among the 26 candidate mRNAs identified in our own samples, 12 showed distinct expression levels between potential NIFTP and other subtypes of PTC in the TCGA dataset. The results confirmed that 8 of the 12 selected mRNAs (KCNC1, PDE1B, NXPH4, ZNF423, MAPT, SRMS, OCLN and IGF2BP3) were upregulated and 4 (AQP5, LYG1, IDO1, and PHYHD1) were downregulated in NIFTP, as shown in FIG. 6.

2-3. Combination of mRNA Markers for Identifying NIFTP

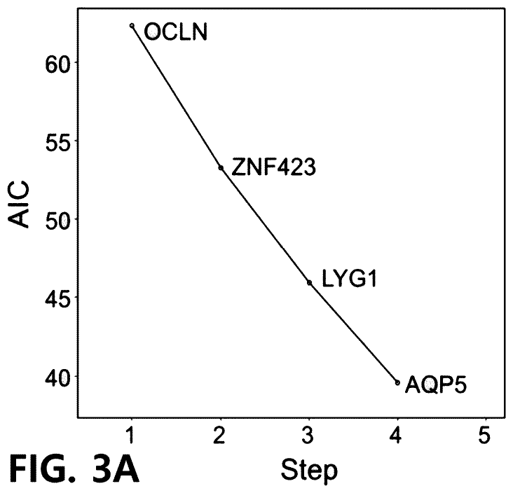

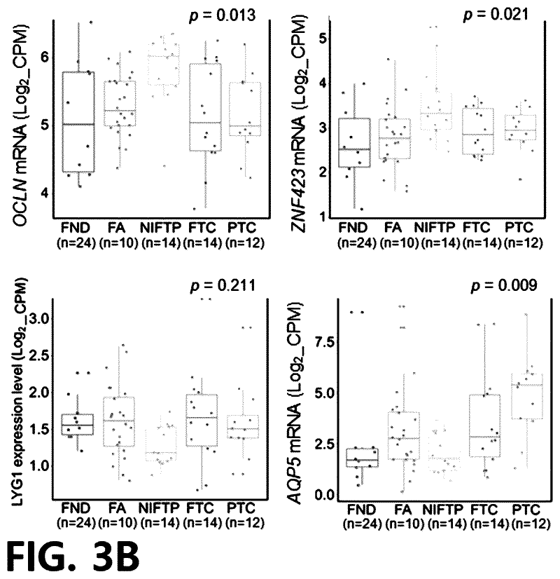

In order to determine the optimal mRNA marker combination for identifying NIFTP, AIC (Akaike Information Criterion) analysis was performed using the top 12 candidate mRNA markers. Ultimately, the inventors of the present disclosure selected occludin (OCLN), zinc Finger Protein 423 (ZNF423), lysozyme g1 (LYG1) and aquaporin 5 (AQP5) mRNA markers based on the expression patterns of the discovery dataset (FIGS. 3A and 3B).

In order to evaluate the accuracy of the above four NIFTP-specific mRNA markers, ROC analysis was performed and AUC values were calculated, and the AUC values for OCLN, ZNF423, LYG1 and AQP5 were 0.80, 0.73, 0.73, and 0.68, respectively (see Table 3).

TABLE 3

| | Cut-off | | | | | |
|---|---|---|---|---|---|---|
| mRNA | value | Sensitivity | Specificity | PPV | NPV | AUC |
| OCLN | 5.436 | 92.9 | 61.7 | 36.1 | 97.4 | 0.802 |
| ZNF423 | 2.718 | 92.9 | 45 | 28.3 | 96.4 | 0.733 |
| LYG1 | 1.568 | 92.9 | 51.7 | 31.0 | 96.9 | 0.727 |
| AQP5 | 3.689 | 100 | 38.3 | 27.5 | 100 | 0.681 |
| Combination of | 23.872 | 100 | 88.3 | 66.7 | 100 | 0.960 |
| four mRNA | | | | | | |
| markers | | | | | | |

In the present disclosure, the inventors of the present disclosure developed Formula 1 below to predict NIFTP using the logistic regression analysis of the above four mRNAs.

$$Prediction score = - 25.12 + ( 3.797 \times expression level of OCLN ) + ( 3.69 \times expression level of ZNF 423 ) + ( - 5.229 \times expression level of LYG 1 ) + ( - 0.756 \times expression level of AQP 5 ) . \qquad < Formula 1 >$$

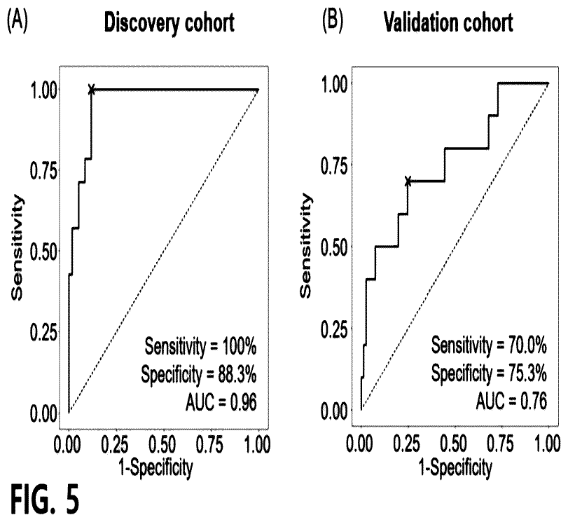

The prediction model had excellent AUC values in both of our cohort (AUC=0.96, (A) of FIG. 5) and the TCGA cohort including potential NIFTP (AUC=0.76, FIG. 5(B)) by applying a reference value of 23.872 to the value derived from Formula 1. The results of this experiment suggest that these four mRNA markers can serve as reliable biomarkers for identifying NIFTP among other thyroid tumors.

That is, if the value calculated according to Formula 1 above is 20 to 25 (preferably 23.872) or higher, it can be predicted that NIFTP has developed.

2-3. Validation of mRNA Markers for Identifying NIFTP in Independent Cohort

The 4 NIFTP-specific mRNA markers identified in the present disclosure were validated in independent samples using qRT-PCR analysis. These samples included normal thyroid tissue, FND, FA, NIFTP, FTC, IEFVPTC and PTC. All four mRNA markers showed significant differential expression across these tumor types (FIG. 5).

Then, in order to evaluate the ability to distinguish NIFTP from other tumor types, logistic regression analysis of these four mRNAs was used to generate Formula 2, which is a prediction equation for NIFTP. The type of sample used in the validation dataset was different from that of the excavation dataset, which was a formalin-fixed paraffin block, and the experimental method for analyzing gene expression levels was also different, and thus, a new prediction equation for NIFTP was generated.

$$Prediction score = - 3.722 + ( 9.95 \times expression level of OCLN ) + ( 70.667 \times expression level of ZNF 423 ) - ( 273.325 \times expression level of LYG 1 ) + ( 2.752 \times expression level of AQP 5 ) . \qquad < Formula 2 >$$

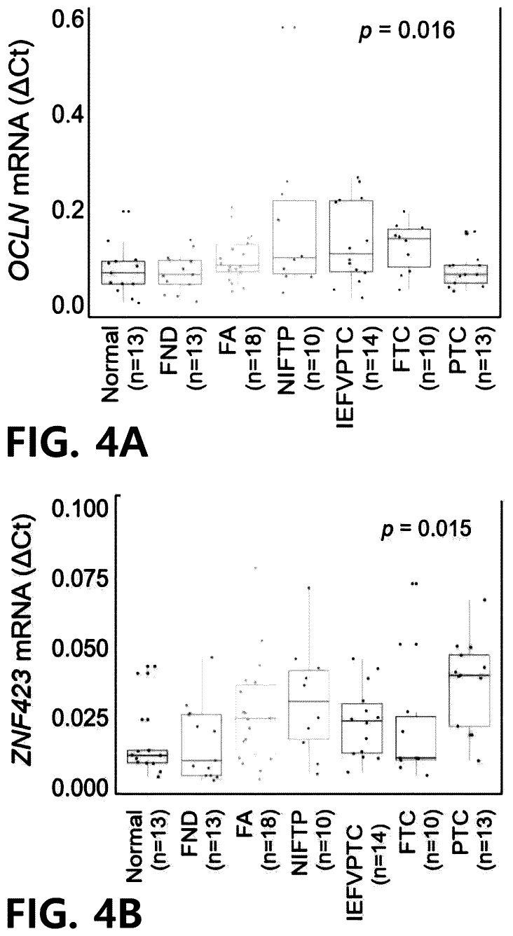

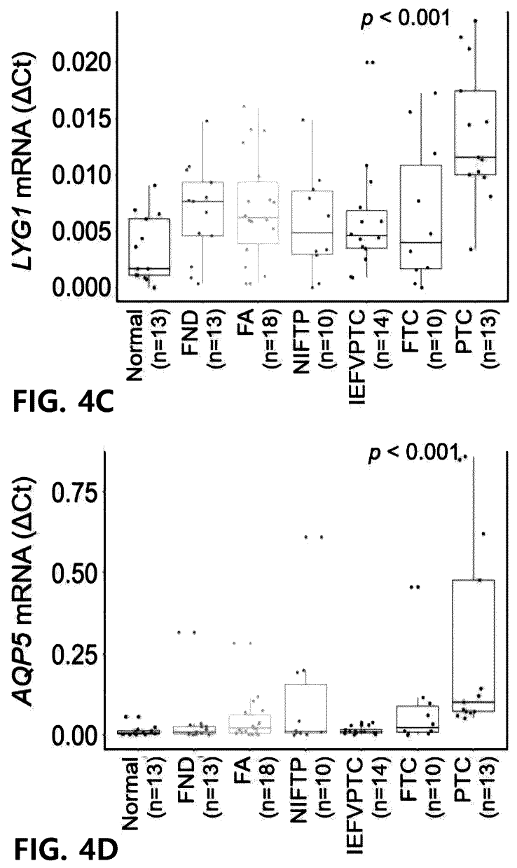

The prediction model showed a high AUC value in the validation data set (AUC=0.757, FIG. 4B). The performance of the model is summarized in Table 4. According to the results, it means that the four mRNA markers of the present disclosure can serve as reliable biomarkers for identifying NIFTP among other thyroid tumors by applying a reference value of 1.374 using Formula 2. According to the present embodiment, when the value calculated according to Formula 2 is 1 to 1.5 (preferably 1.374) or higher, it can be predicted that NIFTP has developed.

TABLE 4

| | Cut-off | | | | | |
|---|---|---|---|---|---|---|
| mRNA | value | Sensitivity | Specificity | PPV | NPV | AUC |
| OCLN | 0.177 | 40 | 88.9 | 30.8 | 92.3 | 0.626 |
| ZNF423 | 0.037 | 50 | 72.8 | 18.5 | 92.2 | 0.598 |
| LYG1 | 0.003 | 50 | 71.6 | 17.9 | 92.1 | 0.575 |
| AQP5 | 0.193 | 30 | 91.4 | 30.0 | 91.4 | 0.510 |
| Combination of | 1.374 | 70 | 75.3 | 25.9 | 95.3 | 0.757 |
| four mRNA | | | | | | |
| markers | | | | | | |

That is, according to the present disclosure, it can be confirmed that OCLN, ZNF423, LYG1 and AQP5 are reliable biomarkers for identifying NIFTP among thyroid tumors.

The above examples are only illustrative of the content of the present disclosure, and the scope of the present disclosure is not limited to the above examples. The examples of the present disclosure are provided to more completely explain the present disclosure to a person having average knowledge in the art.

We claim:

1. A biomarker composition for diagnosing or predicting a prognosis of non-invasive follicular thyroid neoplasm with papillary-like nuclear features (NIFTP), comprising OCLN, ZNF423, LYG1, AQP5 or a combination thereof.

2. The biomarker composition of claim 1, wherein if the expression of the OCLN or ZNF423 gene increases in a sample isolated from a subject, it is predicted or diagnosed that non-invasive follicular thyroid neoplasm with papillary-like nuclear features (NIFTP) has developed.

3. The biomarker composition of claim 1, wherein if the expression of the LYG1 or AQP5 gene decreases in a sample isolated from a subject, it is predicted or diagnosed that non-invasive follicular thyroid neoplasm with papillary-like nuclear features (NIFTP) has developed.

4. A composition for diagnosing or predicting a prognosis of non-invasive follicular thyroid neoplasm with papillary-like nuclear features (NIFTP), comprising an agent for measuring an expression of OCLN, ZNF423, LYG1 or AQP5 gene.

5. The composition of claim 4, wherein the agent for measuring the expression of the gene comprises a peptide, antibody or primer that specifically binds to the gene.

6. A method for diagnosing or predicting a prognosis of non-invasive follicular thyroid neoplasm with papillary-like nuclear features (NIFTP), the method comprising:
a) measuring an expression level of OCLN, ZNF423, LYG1 or AQP5 gene in a sample isolated from a subject;
b) comparing the expression level of the OCLN, ZNF423, LYG1 or AQP5 gene with an expression level of a normal control sample; and
c) determining that non-invasive follicular thyroid neoplasm with papillary-like nuclear features (NIFTP) has developed if the expression level of the OCLN, ZNF423, LYG1 or AQP5 gene measured in the sample isolated from the subject is different from the expression level of the normal control sample.

7. The method of claim 6, wherein the expression level of the OCLN, ZNF423, LYG1 or AQP5 gene is measured by at least any one method selected from the group consisting of Western blot, enzyme-linked immunosorbent assay (ELISA), immunohistochemical staining, immunoprecipitation, immunofluorescence, transcriptome, epigenome and quantitative real-time PCR techniques.

8. The method of claim 6, wherein if the expression of OCLN or ZNF423 gene measured in the sample isolated from the subject in step c) increases compared to a control group, it is determined that non-invasive follicular thyroid neoplasm with papillary-like nuclear features (NIFTP) has developed.

9. The method of claim 6, wherein if the expression of LYG1 or AQP5 gene measured in the sample isolated from the subject in step c) decreases compared to a control group, it is determined that non-invasive follicular thyroid neoplasm with papillary-like nuclear features (NIFTP) has developed.

10. The method of claim 6, wherein the sample isolated in step a) is saliva, urine, tissue, whole blood, serum or plasma.
